(19) United States
(12) Patent Application Publication
(10) Pub. No.: US 2025/0281112 A1
(43) Pub. Date: Sep. 11, 2025

(54) A SURGICAL IMPLANT

(71) Applicants: NewSouth Innovations Pty Limited, Sydney, New South Wales (AU); Macquarie University, North Ryde, New South Wales (AU)

(72) Inventors: Vivek Ananda Sunil Ramakrishna, Sydney (AU); Bellasanty Gangadhara Prusty, Sydney (AU); Ashish Dhar Diwan, Sydney (AU); Subhas Chandra Mukhopadhyay, North Ryde (AU)

(21) Appl. No.: 18/558,006

(22) PCT Filed: May 7, 2021
(86) PCT No.: PCT/AU2021/050423

§ 371 Date: Oct. 30, 2023

(51) Int. Cl. A61B 5/00; A61B 17/00; A61B 17/58; A61B 90/00; A61F 2/30; A61F 2/44; A61F 2/48

(52) U.S. Cl. CPC A61B 5/4851; A61B 17/58; A61B 90/06; A61F 2/4455; A61F 2/482; A61F 2/488; A61B 2017/00022; A61B 2017/00221; A61B 2090/064; A61B 2560/0219; A61B 2560/0462; A61B 2562/0247; A61B 2562/166; A61F 2002/3067

(57) ABSTRACT

The present disclosure is directed to a surgical implant that allows for postoperative monitoring of said implant and an associated surgical site, such as a surgically-repaired joint, and methods of using same.

### TECHNICAL FIELD

The present disclosure relates to devices and methods for use in surgery. In particular, the present disclosure is directed to a surgical implant that allows for postoperative monitoring of said implant and an associated surgical site, such as a surgically-repaired joint, and methods of using same.

### BACKGROUND

In orthopaedic surgery, surgeons and clinicians currently rely on radiographic imaging as a means of post-operatively evaluating the integrity of a surgically repaired region and any associated implant or prosthesis. By way of example, these surgical interventions may be for degenerative joint disease or cases of fracture fixation. Such post-operative imaging typically occurs at intermittent periods after the surgery or at the onset of symptoms arising from complications, such as those arising from the implant itself. This timing, however, is not based on patient-derived post-operative data, but rather the convenience of follow up appointments. Additionally, such imaging modalities can exhibit poor inter-and intra-observer reliability at assessing bone growth and joint integrity and can have an associated cost to the economy, and potentially the patient as well. Further, current imaging techniques generally rely on ionising radiation (e.g., X-Ray or CT scan) to monitor healing, while the associated radiation dose can have negative downstream consequences for patients.

Accordingly, there remains a need for improved devices and methods for post-operatively evaluating the integrity of a surgically repaired region, such as a joint or a fracture, and an associated implant.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is broadly directed to a surgical implant, such as a fracture fixation device or a joint implant, that incorporates or integrates one or more sensors for real-time postoperative monitoring thereof and the associated surgical site. This may facilitate non-invasive monitoring of the surgical implant for the timely diagnosis and preventative management of adverse events associated therewith.

Accordingly, in a first aspect, the present disclosure provides a surgical implant comprising:
one or more sensor elements disposed in and/or on the surgical implant and adapted to measure a force exerted on the surgical implant when implanted in a subject;
a transmitter operably coupled to the one or more sensor elements and adapted to receive a mechanical data representative of the force therefrom and transmit said mechanical data to an external receiver;
a power supply operably coupled to the one or more sensor elements and the transmitter, the power supply adapted to harness energy wirelessly.

Suitably, the one or more sensor elements are selected from the group consisting of a piezoelectric sensor, a piezoresistive sensor, a capacitive sensor and any combination thereof. In some examples, the one or more sensor elements do not include a strain gauge.

In some examples, the one or more sensor elements are operably coupled to a printed circuit board (PCB), such as a thin or flexible PCB or a rigid PCB

In other examples, the one or more sensor elements comprise one or more capacitive sensors not operably coupled to a PCB. For such examples, the one or more capacitive sensors can comprise a biocompatible substrate, such as Polyether-ether-ketone (PEEK), Bioactive glass, polydimethylsiloxane (PDMS), polyimide (PI) and combinations thereof.

Suitably, the one or more sensor elements, the transmitter and/or the power supply are disposed entirely within the surgical implant. In this regard, the one or more sensor elements, the transmitter and/or the power supply are not disposed on an outer surface of the surgical implant so as to not be in contact with a surgical site.

In other examples, the one or more sensor elements, the transmitter and/or the power supply are disposed at least partly on one or more outer surfaces of the implant. For such examples, the one or more sensor elements, the transmitter and/or the power supply are suitably covered by an outer or covering layer to prevent contact with a surgical site.

Suitably, the surgical implant further comprises a processing unit operably coupled to the transmitter and the one or more sensor elements, wherein the processing unit is adapted to process the force measured by the one or more sensor elements into the mechanical data transmittable by the transmitter. In such examples, the processing unit can be disposed in and/or on the surgical implant.

In particular examples, the power supply is adapted to receive or derive energy wirelessly from an external power source.

In certain examples, the power supply is at least partly inductively powered.

Suitably, the surgical implant is switchable between an inactive state, wherein the one or more sensor elements are not measuring the force exerted on the surgical implant, and an active state, wherein the one or more sensor elements are measuring the force exerted on the surgical implant. In some examples, the surgical implant is able to be activated from the inactive state to the active state by positioning of the external power source proximate or adjacent thereto.

In some examples, the power supply is adapted to harvest energy from a subject in which the surgical implant is implanted.

In particular examples, the power supply does not include an incorporated power source, such as a battery unit.

Suitably, the surgical implant is or comprises a fracture fixation device or a joint implant. In some examples, the surgical implant is a spinal implant, such as an interbody fusion cage.

In certain examples, the surgical device does not include a memory unit operably coupled to the one or more sensor elements and/or the processing unit.

In a second aspect, the present disclosure provides a surgical implant system, the system comprising:
(a) a surgical implant comprising:
one or more sensor elements disposed in or on the surgical implant and adapted to measure a pressure and/or a strain exerted on the surgical implant when implanted in a subject;
a transmitter operably coupled to the one or more sensor elements and adapted to receive pressure and/or a strain data therefrom and transmit said data to an external receiver; and
a power supply operably coupled to the one or more sensor elements and the transmitter, the power supply adapted to harness energy wirelessly; and
(b) a control unit comprising a receiver adapted to receive the pressure and/or a strain data from the transmitter of the surgical implant.

In some examples, the control unit further comprises a processor operably coupled to the receiver for processing the mechanical data received therefrom.

In certain examples, the system further includes an external power source adapted for inductively powering the power supply of the surgical implant.

Suitably, the surgical implant is that of the first aspect.

In a third aspect, the present disclosure provides a method of monitoring a surgical implant in a subject, said method including the steps of:
(a) obtaining a mechanical data from one or more sensor elements that are disposed in or on the surgical implant and adapted to measure a force exerted thereon;
(b) transmitting the mechanical data by a transmitter operably coupled to the one or more sensor elements and adapted to receive data therefrom to a receiver external to the surgical implant and the subject; and
(c) powering the one or more sensor elements and the transmitter with a power supply operably coupled thereto, wherein the power supply is adapted to harness energy wirelessly.

Suitably, the present method further includes the step of determining the force exerted on the surgical implant based at least in part on the mechanical data received by the receiver.

In certain examples, the present method further includes the step of determining an integrity, alignment, stability and/or positioning of the surgical implant in the subject based at least in part by the mechanical data received by the receiver.

In other examples, the surgical implant is implanted within a joint of the subject and step (a) includes obtaining the mechanical data during movement of the joint, such as through a range of motion of the joint.

Suitably, said method is performed at a plurality of time points, such as at first and second time points.

In a fourth aspect, the present disclosure relates to a method of monitoring healing of a bone fracture in a subject, wherein the bone fracture is fixed with a surgical implant, said method including the steps of:
(a) obtaining mechanical data from one or more sensor elements that are disposed in or on the surgical implant and adapted to measure a force exerted thereon;
(b) transmitting the mechanical data by a transmitter operably coupled to the one or more sensor elements and adapted to receive data therefrom to a receiver external to the surgical implant and the subject;
(c) powering the one or more sensor elements and the transmitter with a power supply operably coupled thereto, wherein the power supply is adapted to receive or harvest energy wirelessly; and
(d) processing the mechanical data to determine a degree of healing of the bone fracture in the subject.

In particular examples, said method is performed at first and second time points and detecting a change, alteration or modulation in the mechanical data between the first and second time points indicates or correlates with the degree of healing of the bone fracture in the subject.

In a fifth aspect, the present disclosure provides a method for monitoring fusion of two adjacent vertebrae with a surgical implant implanted therebetween, said method including the steps of:
(a) obtaining mechanical data from one or more sensor elements that are disposed in or on the surgical implant and adapted to measure a force exerted thereon;
(b) transmitting the mechanical data by a transmitter operably coupled to the one or more sensor elements and adapted to receive data therefrom to a receiver external to the surgical implant and the subject;
(c) powering the one or more sensor elements and the transmitter with a power supply operably coupled thereto, wherein the power supply is adapted to receive or harvest energy wirelessly; and
(d) processing the mechanical data to determine a degree of fusion of the two adjacent vertebrae in the subject.

In some examples, the method may further include the initial step of implanting the surgical implant between the two adjacent vertebrae.

In particular examples, said method is performed at first and second time points and detecting a change, alteration or modulation in the mechanical data between the first and second time points indicates or correlates with a degree of fusion of the two adjacent vertebrae in the subject.

In a sixth aspect, the present disclosure describes a method for determining an integrity, alignment, stability and/or positioning of a surgical implant in a subject, said method including the steps of:
(a) obtaining mechanical data from one or more sensor elements that are disposed in or on the surgical implant and adapted to measure a force exerted thereon;
(b) transmitting the mechanical data by a transmitter operably coupled to the one or more sensor elements and adapted to receive data therefrom to a receiver external to the surgical implant and the subject;
(c) powering the one or more sensor elements and the transmitter with a power supply operably coupled thereto, wherein the power supply is adapted to receive or harvest energy wirelessly; and
(d) processing the mechanical data to determine the integrity, alignment and/or positioning of the surgical implant in the subject.

In a seventh aspect, the present disclosure relates to a method for monitoring movement of a subject's joint previously implanted with a surgical implant, said method including the steps of:
(a) obtaining mechanical data from one or more sensor elements that are disposed in or on the surgical implant and adapted to measure a force exerted thereon during movement of the joint;
(b) transmitting the mechanical data by a transmitter operably coupled to the one or more sensor elements and adapted to receive data therefrom to a receiver external to the surgical implant and the subject;
(c) powering the one or more sensor elements and the transmitter with a power supply operably coupled thereto, wherein the power supply is adapted to receive or harvest energy wirelessly; and
(d) processing the mechanical data to determine the force exerted on the surgical implant during movement of the joint.

Referring to the methods of the third to the seventh aspects, step (c) suitably includes positioning an external power source adapted to inductively power the power supply proximate or adjacent the surgical implant.

For the third to the seventh aspects, said methods can include the further step of generating a representation of the mechanical data received by the transmitter and displaying the representation on a display.

In relation to the methods of the third to the seventh aspects, the surgical implant is suitably that of the first aspect.

Suitably, the surgical implant of the first aspect or the surgical system of the second aspect are suitable for use in the method of the third to seventh aspects.

In an eighth aspect, the present disclosure describes a non-transitory computer-readable storage medium whose stored contents configure a computing system to perform the method of the third to seventh aspects.

In a ninth aspect, the present disclosure relates to an interbody cage, the interbody cage comprising:
one or more sensor elements adapted to measure a force exerted on the interbody cage when implanted in a subject;
a transmitter operably coupled to the one or more sensor elements and adapted to receive a mechanical data representative of the force therefrom and transmit said mechanical data to an external receiver;
a power supply operably coupled to the one or more sensor elements and the transmitter, the power supply adapted to harness energy wirelessly;
wherein the one or more sensor elements, the transmitter and/or the power supply are disposed in and/or on the interbody cage.

Any embodiment herein shall be taken to apply mutatis mutandis to any other embodiment unless specifically stated otherwise.

It will be appreciated that the indefinite articles “a” and “an” are not to be read as singular indefinite articles or as otherwise excluding more than one or more than a single subject to which the indefinite article refers.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

In order that the present disclosure may be readily understood and put into practical effect, reference will now be made to the accompanying illustrations, wherein like reference numerals are used to refer to like elements.

FIG. 1: is a perspective view of an example of a cage body of an interbody cage.

FIG. 2: is a perspective view of an example of a cage lid of an interbody cage for use with the cage body of FIG. 1.

FIG. 3: provides top-down (A) and cross-sectional (B) views of a flexible sensor for use with a surgical implant described herein.

FIG. 4: is a schematic of an example of a surgical system of the present disclosure.

FIG. 5: is a sagittal plane view of computed tomography scans segmented into anatomical regions of interest in preparation for modelling.

FIG. 6: is a sagittal plane view of the interbody cage implanted between L4 and L5 vertebrae depicting separation of the superior endplate and the implant surface during motion in early stages of bone healing (a) and bonded contact between the superior endplate and the implant surface during motion after full bony fusion (b).

FIG. 7: is a screen capture demonstrating the modelling of annulus fibrosus fibres in the intervertebral disc for modelling, showing a single ring of criss-cross fibres (a), the four concentric rings of fibres embedded in the ground substance of the annulus fibrosus with the nucleus pulposus (b), the four concentric rings of fibres without the ground substance (c), and the four concentric rings of fibres embedded in the annulus fibrosus without the nucleus pulposus.

FIG. 8: is a bar chart of compressive stress on the interbody cage in flexion and extension for varying graft stiffnesses, where error bars represent standard deviation.

FIG. 9: is a bar chart of normalised percentage change in force on the interbody cage in flexion and extension for varying graft stiffnesses.

FIG. 10: is a mid-axial cut view of compressive stress on the interbody cage in flexion and extension for varying graft stiffnesses.

FIG. 11: is a bar chart of compressive stress on the graft in flexion and extension for varying graft stiffnesses, where error bars represent standard deviation.

FIG. 12: is a bar chart of normalised percentage change in force on the graft in flexion and extension for varying graft stiffnesses.

FIG. 13: is a bar chart of compressive load-sharing between the cage and graft as a percentage of total compressive stress on the construct for varying graft stiffnesses in flexion and extension.

FIG. 14: is a mid-axial cut view of compressive strain on the cage-graft construct (footprint) in flexion and extension for varying graft stiffnesses.

FIG. 15: is a bar chart of anteriorly directed force on the interbody cage for varying graft stiffnesses with unfused contact in flexion and extension.

### DETAILED DESCRIPTION

The present disclosure provides a surgical device or implant for use during fracture fixation, joint replacement surgery or joint fusion surgery that can advantageously provide real-time biomechanical data about the surgically repaired area to clinicians post-operatively. This data can indicate to clinicians, for example, the mechanical integrity, alignment, position and/or stability of an implant as well as the degree of healing, such as bone growth, or fusion associated with the surgically repaired area.

Accordingly, in one broad form the present disclosure relates to a surgical implant comprising:
one or more sensor elements disposed in and/or on the surgical implant and adapted to measure a force exerted on the surgical implant when implanted in a subject;
a transmitter operably coupled to the one or more sensor elements and adapted to receive a mechanical data representative of the force therefrom and transmit said mechanical data to an external receiver;
a power supply operably coupled to the one or more sensor elements and the transmitter, the power supply adapted to derive, receive or harvest energy wirelessly.

While the principles illustrated herein are based on methods of providing surgical devices for humans, this surgical implant may also be extended to other mammals such as livestock (e.g. cattle, sheep), performance animals (e.g. racehorses) and domestic pets (e.g. dogs, cats), although without limitation thereto.

As used herein the term “anterior” refers to the front side from the perspective of the patient, while the term “posterior” refers the backside from the perspective of the patient. Further, as used herein, the term “superior” means closer to the head of the patient and “inferior” means closer to the feet of the patient.

The surgical implant described herein may be made of any material suitable for implantation in a subject. Exemplary materials include polyaryletherketone, titanium, stainless steel, cobalt-chromium alloy, titanium alloy (such as Ti-6Al-4V titanium alloy or Ti-6Al-7Nb titanium alloy), .porous tantalum, ceramic material (such as silicon nitride (Si3N4)), or an implantable-grade composite material (such as implantable-grade polyaryletherketone with filler, implantable-grade polyetheretherketone with filler, implantable-grade polyetheretherketone with carbon fiber, or implantable-grade polyetheretherketone with hydroxyapatite, implantable-grade polyetheretherketone without composite fillers). Suitably, the surgical implant is sterile.

Suitably, the sensor elements or sensors described herein may be any sensor capable of detecting and/or measuring a force, such as a mechanical force (e.g., pressure, strain, stress, tensile forces, compressive forces, shear forces etc) imparted thereon. In some examples the sensor elements are microelectromechanical sensors (MEMS). In further examples, the sensor elements are selected from a piezoelectric sensor, a piezoresistive sensor, a capacitive sensor and any combination thereof. As used herein, the sensor elements can have any suitable size and/or shape, as the present disclosure is not limited in this respect. In one example, the one or more sensor elements do not include a strain gauge.

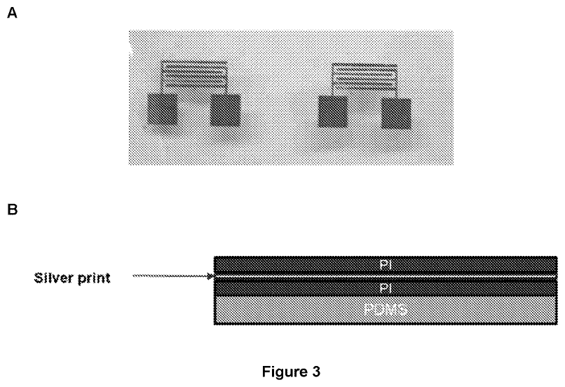

In some examples, however, the one or more sensor elements are or comprise a flexible sensor. In this regard, flexible sensors can be multi-layer laminated structures including, for example, a flexible substrate layer (e.g., a biocompatible polymeric material, PDMS, PI), an electrode layer comprising a conductive pattern of conductive material (e.g., silver, gold, titanium, an organic conductive material) overlying the substrate layer and optionally one or more flexible covering or insulating layers (e.g., PI) overlying the electrode layer. An exemplary flexible sensor is provided in FIG. 3. The flexible sensor may be fabricated by any means known in the art. In particular examples, the flexible sensor may be fabricated, at least in part by, moulding and deposition, photolithography and/or aerosol jet printing.

In certain examples, the sensor elements are adapted to evaluate whether or not a subject is exerting excessive forces on a surgically repaired area, such as a joint, that contains the surgical implant. In this regard, the sensor elements can be used to evaluate if the subject is using reasonable restraint in their movements, such as rehabilitation exercises, of the surgically repaired area or joint.

In other examples, the sensor elements are adapted to evaluate forces across a bone fracture or in the fusion of two adjacent vertebrae so as to monitor healing thereof. By way of example, an alteration, such as a decrease, in the force exerted on the surgical implant over time (e.g., between first and second time points) may indicate or correlate with increasing maturing bone growth or callus formation at the fracture site or between the two adjacent vertebrae. Alternatively, an alteration, such as an increase or no change, in the force exerted on the surgical implant over time may indicate, for example, a non-union, a delayed union, pseudarthrosis, subsidence or a further fracture.

In further examples, the sensor elements are adapted to evaluate an integrity, alignment, stability and/or positioning of a surgical implant in a subject. By way of example, a modulation, such as an increase or decrease, in the force measured, such as between first and second time points, in one or more regions of the surgical implant can be indicative of a loss of integrity of the implant and/or misalignment, malpositioning, migration, subsidence and/or instability of the surgical implant post-operatively, which may require the further surgical step of replacing, re-aligning or re-positioning the surgical implant. In particular examples, the sensor elements are adapted to detect migration or subsidence of a surgical implant, such as an interbody cage.

In some examples, the surgical implant described herein further comprises a processing unit, such as a processor, microprocessor or microchip, operably coupled to the transmitter and the one or more sensor elements. Suitably, the processing unit is configured to convert signals or data from the one or more sensor elements that is representative of the forces detected or sensed thereby into mechanical data that is of a format suitable for transmission by the transmitter (e.g., converting an analog signal to a digital signal). It will be understood that the processing unit can be disposed on an outer surface of the surgical implant or more preferably disposed entirely within the surgical implant.

Further to the above, the mechanical data measured or acquired by one or more of the sensor elements of the surgical implant can then be transmitted by the transmitter by any wireless or telemetry means, protocol or method known in the art to an external or remotely located processor and/or computing device. In typical examples, the mechanical data can be transmitted by way of any conventional data transmission protocol as are known in the art, such as BlueTooth, Wi-Fi or the like. In some examples, the transmitter is a transceiver that is capable of transmitting and receiving signals.

Once processed by the external processor and/or the computing device, the mechanical data can then be transmitted to a display operably coupled or connected thereto. In particular examples, a force exerted on the sensor elements is converted to mechanical data by the processing unit and sent to the external processor and/or computer device by the transmitter, where it is processed and converted to a visual representation, such as a curve or a graph by software therein. This visual representation of the mechanical data can then be displayed on the display. In some examples, the computing device includes a memory unit adapted to store the mechanical data from the one or more sensor elements for future analysis and graphical display by the surgeon if required.

Suitably, the one or more sensor elements, the transmitter and/or the power supply are disposed entirely within the surgical implant, such as in one or more internal cavities or spaces therein. In this regard, the one or more sensor elements, the transmitter and/or the power supply may be hermetically sealed within the surgical implant. In one example, the one or more sensor elements are disposed entirely within the surgical implant, whilst the the transmitter and the power supply are at least partly disposed on the surgical implant, such as on an outer surface thereof. In another example, the one or more sensor elements and the transmitter are disposed entirely within the surgical implant, whilst the the power supply is at least partly disposed on the surgical implant, such as on an outer surface thereof. In alternative examples, the one or more sensor elements, the transmitter and/or the power supply are disposed at least partly on one or more outer surfaces of the surgical implant. In this regard, the one or more sensor elements (and associated circuitry for operably coupling with the power supply and the transmitter) can be printed, such as aerosol jet printed, directly into an internal layer or surface of the surgical implant, such as during manufacture or 3D printing of the surgical implant. In particular examples, inclusion of the one or more sensor elements (and optionally any associated circuitry, the transmitter, the power supply and/or the processor) within the surgical implant of the present disclosure may include an integrated manufacturing approach that may include, for example, combining aerosol jet printing and 3D printing. In examples in which the one or more sensor elements, the transmitter and/or the power supply are disposed at least partly on one or more outer surfaces of the surgical implant, preferably the surgical implant includes a cover layer extending over or overlying the one or more sensor elements, the transmitter and/or the power supply so as to protect or shield these components from direct contact with the surgical site of the subject.

It is further envisaged that the surgical implant described herein may include one or more further sensor elements to utilise with the aforementioned sensor elements, such as temperature sensors (e.g., a thermocouple), accelerometers, strain sensors (e.g., a strain gauge,) position sensors, chemical sensors, a volume sensor, a variable resistance sensor, a gyrometer, an acoustic sensor and the like. Such further sensor elements may be positioned adjacent or remote from the one or more sensor elements in and/or on the surgical implant as required.

The surgical implant provided herein may utilise one or more power management strategies. Such strategies may include implanted power sources, harvestable power sources and/or inductive power sources. In particular examples, however, the surgical implant does not include an incorporated or implanted power source, such as a battery unit or a capacitor. In alternative examples, the surgical implant includes a power source that may be chargeable by the power supply.

In some examples, the power supply is adapted to harvest or derive energy wirelessly, such as a power harvester or an energy scavenging device, such as from vibrations or motion from in vivo patient movements and/or in vivo temperature differentials. In some examples, the power supply includes a motion powered piezoelectric or electromagnetic generator.

In other examples, the power supply is adapted to receive energy wirelessly, such as from an external power source. In this regard, the power supply, and hence the various components of the surgical implant described herein, can be at least partly inductively powered by an inductive power source. Inductive power sources include inductive coupling systems and Radio Frequency (RF) electromagnetic fields. In those examples in which the power supply is inductively powered by an external inductive power source, the implant can comprise an antenna or a coil that can function as an induction coil for receiving energy by a radio frequency (RF) signal or a magnetic field. The induction coil can then direct an electric current derived from the RF signal or the magnetic field to the one or more sensor elements, the transmitter and the processing unit. A magnetic field can be applied externally with one or more magnets, for example, of an MRI instrument or other magnetic or electromagnetic devices to induce an electrical current in the induction coil and the power supply.

In view of the foregoing, the surgical implant can advantageously be switchable between active and inactive states. By way of example, the surgical device can be maintained in an inactive or passive state for a period of time once implanted. In the inactive state, the power supply is suitably not receiving or harvesting any energy (or minimal energy) wirelessly to power the one or more sensor elements, the processing unit and the transmitter. Accordingly, the one or more sensor elements are not detecting or measuring any forces imparted on the surgical implant and no mechanical data is being transmitted to an external processor in this inactive state.

When required by a clinician or the like, the surgical implant can be activated or switched to the active state by positioning an external inductive power source within a sufficient distance of the surgical implant so as to inductively power the induction coil of the power supply. The power supply can now power the one or more sensor elements, the processing unit and the transmitter electrically coupled thereto. This arrangement advantageously allows for the simple turning on and off of the ability of the surgical implant to generate and transmit mechanical data as required by a user rather than relying on continuous or near continuous data generation and transmission.

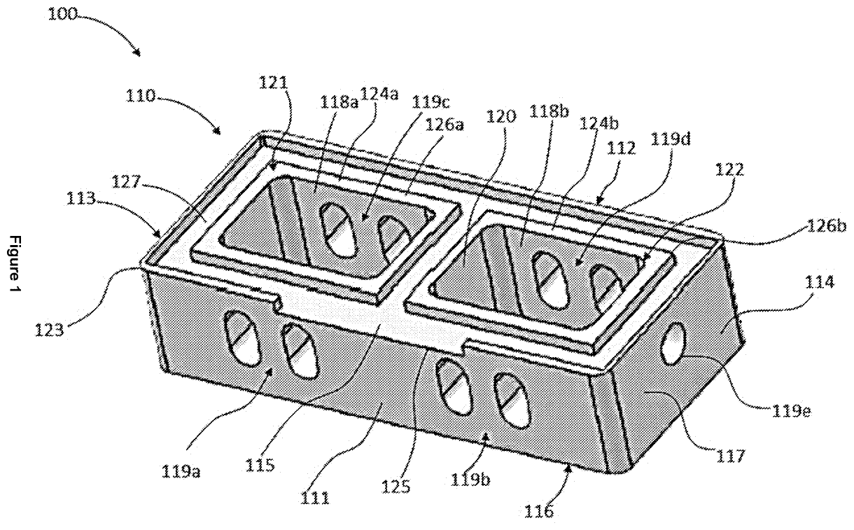

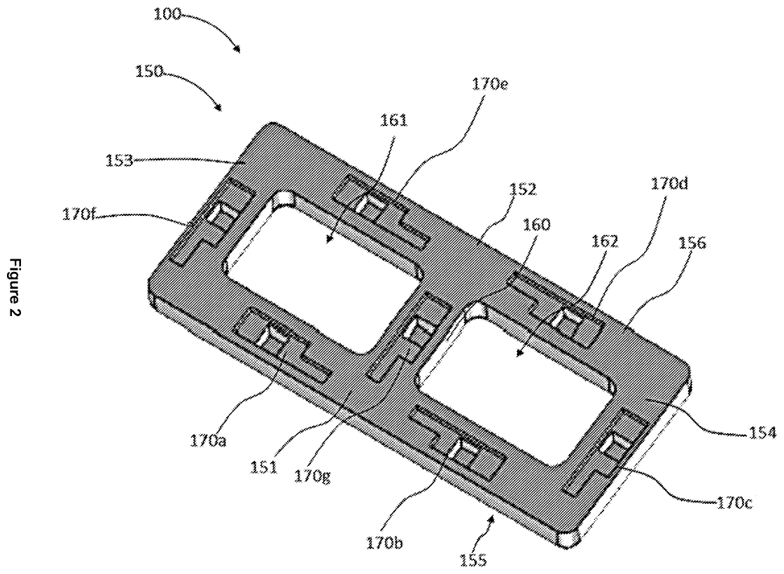

An example of an interbody fusion device or interbody cage 100 is illustrated in FIGS. 1 and 2. While the surgical devices and implants described herein are exemplified by an interbody cage 100 suitable for use in spinal fusion surgery, the present disclosure has general applicability to surgical implants for all types of joints (e.g., knees, elbows, shoulders, wrists and fingers) and replacement surgery thereof, as well as fracture fixation devices as are known in the art. Accordingly, the surgical implant can be a joint implant, such as a knee implant (e.g., a tibial component or tray, a spacer, a femoral component, a patellar component), a shoulder implant (a glenoid component, a spacer, a humeral component or stem) or a hip implant (e.g., a femoral component, a liner or spacer, an acetabular component). In alternative examples, the surgical implant is a fracture fixation device, such as a fixation plate.

The interbody cage 100 comprises a generally cuboidal shaped cage body 110 of appropriate dimensions to fit between adjacent vertebrae of a patient. It is envisaged, however, that the cage body 110 may be of any three dimensional shape, such as oblong, arcuate, concave, convex etc as is known in the art, to match patient anatomy and the particular vertebrae intended to be fused. The interbody cage 100 can be implanted, for example, in the cervical spine, the thoracic spine, or the lumbar spine of a subject. Exemplary adjacent vertebral bodies suitable for implantation of the interbody cage 100 include adjacent vertebral bodies from among C2-T1 vertebrae, adjacent vertebral bodies from among T1-T12 vertebrae, adjacent vertebral bodies of L4-L5 vertebra, and adjacent vertebral bodies of L5-S1 vertebrae, among others.

It is intended that the interbody cage 100 described herein can be used in a variety of spinal interbody fusion applications. Exemplary interbody cages include an anterior lumbar interbody fusion (ALIF) interbody cage, a posterior lumbar interbody fusion (PLIF) interbody cage, a lateral interbody cage, a direct lateral interbody fusion (DLIF) interbody cage, a transforaminal lumbar interbody fusion (TLIF) interbody cage, an extreme lateral interbody fusion (XLIF) interbody cage, and a cervical interbody cage, among others.

The cage body 110 includes an elongate and opposed pair of planar side walls 111,112 that are positioned anteriorly and posteriorly and define a longitudinal axis of the interbody cage 100. The cage body 110 further contains a pair of opposed planar end walls 113,114 that are positioned laterally and extend perpendicularly between and interconnecting respective ends of the side walls 111,112. The end walls 113,114 are of shorter dimensions than the side walls 111,112 so as to define a short axis of the interbody cage 100. Together, the side walls 111,112 and the end walls 113,114 define upper and lower surfaces 115,116 of the cage body 110 and an outer surface 117 extending therearound.

As can be observed in FIG. 1, a central inner wall 120 that is parallel to the end walls 113,114 extends perpendicularly between inner central portions of the side walls 111,112 to define first and second inner spaces 121,122. Accordingly, the first and second inner spaces 121,122 are defined by respective inner surfaces 118a-b of the side, end and inner walls 111-114,120 and are configured for receiving graft material therein so as to initiate fusion of the vertebrae between which the interbody cage 100 is implanted. In the example provided, the side walls 111,112, the end walls 113,114 and the inner wall 120 are substantially planar, although it is appreciated that these walls may instead be modified so as to be concave, convex, corrugated etc as is known in the art.

From FIG. 1, each of the side walls 111,112 contain two pair of opposed and adjacent slots or apertures 119a-d, each pair spaced apart along the side walls 111,112 and extending therethrough so as to open into the respective first and second inner spaces 121,122. Similarly, each of the end walls 113,114 include a single slot or aperture 119e (and not shown) centrally positioned therein and extending therethrough so as to open into the respective first and second inner spaces 121,122.

As illustrated in FIG. 1, the cage body 110 further includes an outer lip 123 that extends upwardly and outwardly from the upper surface 115 and extends substantially around an outer edge 125 thereof, except for a central anterior portion that remains flush with the upper surface 115. The cage body 110 further contains a pair of inner lips 124a-b that are of similar dimensions to the outer lip 123 and extend upwardly and outwardly from the upper surface 115 and extend around an inner edge 126a-b defined by the first and second spaces 121,122. By virtue of this arrangement, the inner lips 124a-b, the outer lip 123 and the upper surface 115 of the cage body 110 define an open channel 127 therebetween. Although not shown in FIG. 1, the channel 127 is of suitable dimensions for receiving at least partly therein one or more components of a sensor module, such as one or more sensor elements, a power supply (e.g., an induction coil), a processor (e.g., a microprocessor), a PCB and circuitry for operably or electrically coupling said components (not shown).

As shown in FIG. 2, the interbody cage 100 further includes a cage top or lid 150 adapted to be engaged or fastened thereto. The cage lid 150 defines an outer lid surface 155 and an inner lid surface 156, the inner lid surface 156 to be facing and proximate the upper surface 115 of the cage body 110 when engaged therewith. Similar to the cage body 110, the cage lid 150 is of a generally cuboidal shape and includes a pair of opposed side portions 151,152 and a pair of opposed end portions 153,154. Further, an inner portion 160 is disposed centrally in the cage lid 150 and extends perpendicularly between the respective side portions 151,152 and parallel to the end portions 153,154 to define cuboidal open portions 161,162 therebetween. The side portions 151,152, the end portions 153,154 and the inner portion 160 are of appropriate dimensions so as to substantially overlie the side walls 111,112, the end walls 113,114 and the inner wall 120 respectively of the cage body 110 when engaged therewith.

Referring to FIG. 2, the cage lid 150 includes seven spaced apart recesses 170a-g disposed in the inner lid surface 156 thereof. In this regard, each of the end portions 153,154 and the inner portion 160 includes a single recess 170c,f,g, whilst each of the side portions 151,152 include a pair of recesses 170a,b,d,e spaced apart therealong. Each of the recesses 170a-g are of suitable dimensions for receiving a sensor element (not shown) therein. By virtue of this arrangement, each sensor element (and any associated circuitry, PCB etc) is to be hermetically sealed within a space defined by the open channel 127 of the cage body 110 and the recesses 170a-g of the cage lid 150 upon engagement of the cage lid 150 to the cage body 110. To this end, the sensor elements (not shown) of the interbody cage 100 are configured to assess any mechanical forces exerted on the outer lid surface 155 of the cage lid 150. Although not shown in the present example, a similar arrangement with respect to the lower surface 116 of the interbody cage 100 is contemplated, such that mechanical forces at both the superior and inferior vertebral bodies adjacent the interbody cage 100 may be assessed.

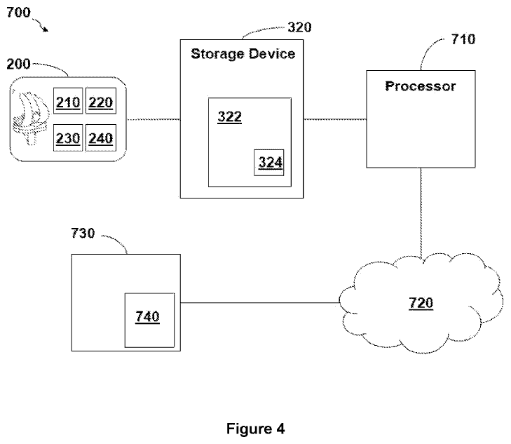

FIG. 4 illustrates an apparatus or system 700 according to one example of the present disclosure. The apparatus 700 comprises a processor 710 in communication with a surgical implant 200 and a storage device 320. The surgical implant 200 can comprise a sensor element 210, a transmitter 220, a power supply 230 which can derive energy wirelessly and a processing unit 240, such as those previously described, operably coupled together therein. Suitably, the processing unit 240 processes those forces sensed by the sensor element 210 into a mechanical data, which is suitable for transmission by the transmitter 220 to the processor 710 and/or the storage device 320. The mechanical data from the surgical implant 200 may further be transmitted or received over a network via the communications network 720 utilising any one of a number of well-known transfer protocols (e.g., HTTP, UDP, TCP, USSD, FTP). It is further envisaged that the system 700 may include an external amplifier or transceiver (not shown), such as included in a wearable device, that is adapted to receive the mechanical data from the transmitter 220 and amplify or re-transmit this signal to the processor 710 and/or the storage device 320.

The processor 710 then generates one or more reports 740 based on input of the mechanical data transmitted wirelessly from the surgical implant 200, which for the present example is a tibial component 200 for use in knee replacement surgery. It is contemplated, however, that alternative surgical implants, as are known in the art, may also be utilised for the present system 700.

The processor 710 can, for example, form part of a server which comprises the storage device 320 or be a separate computing device that is in communication with the storage device 320. In particular examples, the processor forms part of a computer, such as be a personal computer (PC), a tablet PC, a set-top box (STB), a Personal Digital Assistant (PDA), a cellular telephone, a web appliance, a network router, switch or bridge, or any computer capable of executing a set of instructions (sequential or otherwise) that specify actions to be taken by that computer, as are known in the art. The term “computer” shall also be taken to include any collection of computers that individually or jointly execute a set (or multiple sets) of instructions to perform any one or more of the methodologies discussed herein. The computer can operate as a standalone device or may be connected (e.g. networked) to other computers. In a networked deployment, the computer may operate in the capacity of a server, as described earlier, or a client computer in a server-client network environment, or as a peer computer in a peer-to-peer (or distributed) network environment.

The processor 710 provides a graphical user interface (GUI) 730 comprising the one or more reports 740 via a communications network 720, for example, to a computing device of a user or administrator. The one or more reports can include one or more metrics or readouts for, for example, monitoring the surgical implant 200, monitoring healing of a bone fracture, monitoring fusion of two adjacent vertebrae, determining an integrity, alignment, stability and/or positioning of the surgical implant 200, and/or monitoring movement of a subject's joint, as previously described, based on the mechanical data transmitted by the surgical implant 200. In some examples, the one or more reports include one or more visualisations or classifications of the aforementioned metrics or readouts, as hereinbefore described, generated based on the mechanical data transmitted from the surgical implant 200 and the GUI 730 can comprise one or more controls to select the one or more visualisations to be displayed.

The storage device 320 can comprise a computer memory 322 which can be, for example, a computer readable medium (e.g., software embodying or utilised by any one or more of the methodologies or functions described herein), such as, one or more hard disk drives or solid state drives. The computer memory 322 stores the mechanical data transmitted by the surgical implant 200. The computer memory 322 can also comprise computer readable code components 324 that when selectively executed by the processor 710 implements one or more aspects of the present disclosure, such as, generating aspects of the GUI 730 and providing the GUI 730 via the communications network 720.

In one further aspect, the present disclosure resides in a computer-readable medium, such as a non-transitory computer-readable medium, having stored thereon a computer program, which, when executed by a computer, causes the computer to perform the method of any one of the aforementioned aspects.

### EXAMPLE 1

In the present example, the present inventors generated modelling data of graft stiffness changes over time for an interbody cage implanted within the intervertebral space that may be helpful in monitoring the fusion of two adjacent vertebrae with a surgical implant, and more particularly, an interbody cage of the present disclosure.

### Method

### Image Segmentation and Model Generation from Computed Tomography Data

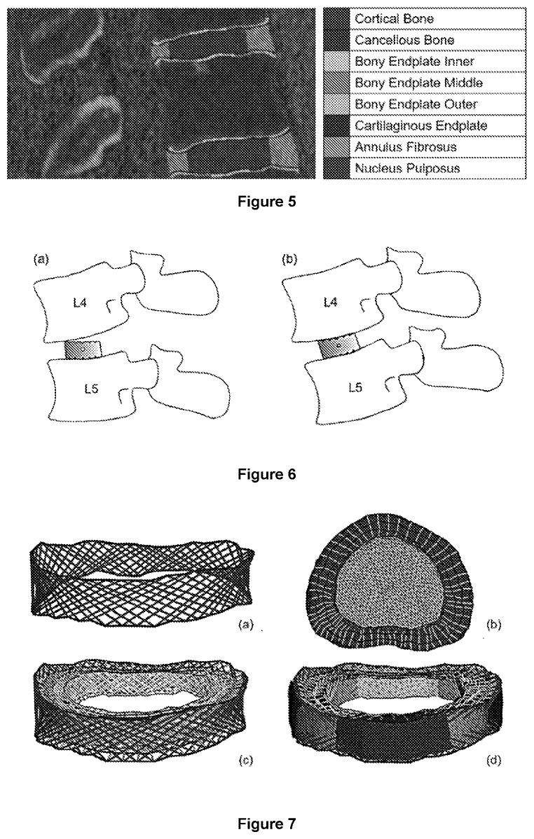

High-resolution thoracolumbosacral spine Computed Tomography (CT) data (1291 axial cuts, 512×512 pixel resolution, 0.30 mm slice thickness) from an anonymised asymptomatic male subject (55 years old) were obtained in DICOM (Digital Imaging and Communications in Medicine) file format from Southern Radiology Miranda (Sydney, Australia). The CT data were imported into Materialise Mimics image processing software (Materialise NV 2018b) for segmentation into anatomical regions of interest for L1-S1 (FIG. 5).

The nucleus pulposus was assumed to occupy 43% of the total intervertebral disc volume¹. Further segmentation was undertaken on the annulus fibrosus into five regions for ease of assigning regional stiffness variation according to Schmidt et al. (2006)². Similarly, the bony endplates were modelled for stiffness variation in three regions according to Denoziére & Ku (2006) with equal radial width and thickness of 0.6 mm³. The cartilage endplate was segmented with a thickness of 0.3 mm⁴.

The segmented regions were digitally stitched to generate a surface mesh of 3-noded triangle elements in Materialise 3-Matic (Materialise NV 2018a). The 3D model file (STL) of the XLIF cage (22×50×10 mm, 0° lordosis) was imported into Materialise 3-Matic and embedded within the L4-L5 intervertebral space using a Boolean operation. Subsequent re-meshing and triangle quality adjustment enabled successful 3D volumetric mesh generation. The 3D volumetric mesh was imported in Nastran file format (.nas) into Strand7 (vers. 2.4.6, Strand7 Pty. Ltd., Sydney, Australia) finite element (FE) modelling software for pre-processing.

### Modelling Temporal Graft Stiffness Changes

Across all the FE models, the interbody cages remained bonded to the L5 superior endplate; however, two states of contact were modelled between the L4 inferior endplate and superior cage and graft surface. Unbonded contact represented immature fusion progression and incomplete union between the two surfaces, which was modelled using Normal Contact elements in Strand7 that allowed for simultaneous lift-off and compressive contact on different regions of the superior cage surface during simulated bending motions (FIG. 6). Bonded contact represented bony union through the cage-graft construct, from the L4 inferior endplate to the L5 superior endplate. Five unique graft stiffnesses were modelled in the unbonded state and two unique graft stiffnesses in the bonded state.

Graft material variation in the unbonded state represented temporal stiffening from the soft callus (SC) formation stage to the solid graft (SG) state, simulated with silicone and poly (methyl methacrylate) (PMMA) respectively. Between the two endpoints, three intermediate stiffness stages were modelled for which material properties were obtained using a unit cell approach. Stress-strain curves were obtained for 25%, 50%, and 75% volume occupancy of PMMA in a silicone unit cell, which were assigned as material properties to temporal stages St1, St2, and St3, respectively. Partial fusion (PF) was modelled with bonded contact and a cancellous bone fusion mass, representing progressive bone formation with attachment to the endplates. Similarly, full fusion (FF) represented the final stage of bone healing, consisting of a cortical bone fusion mass bonded to the endplates.

### Modelling Annulus Fibres and Ligaments

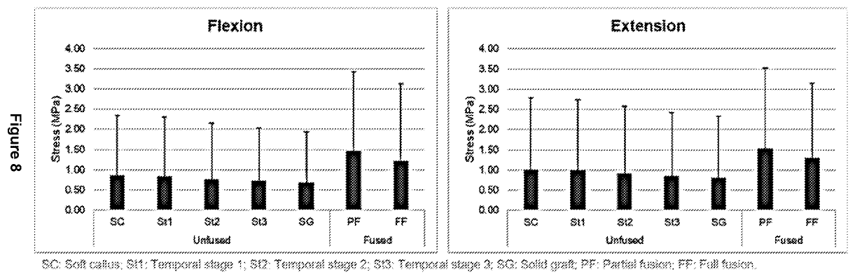

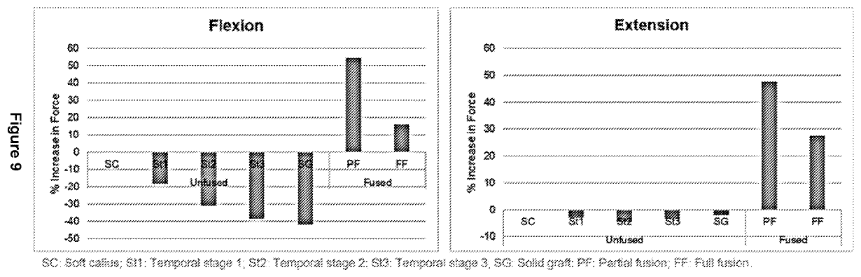

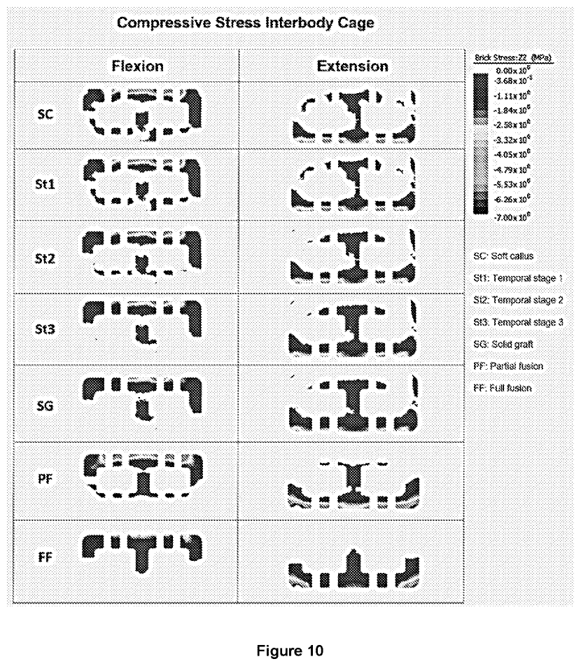

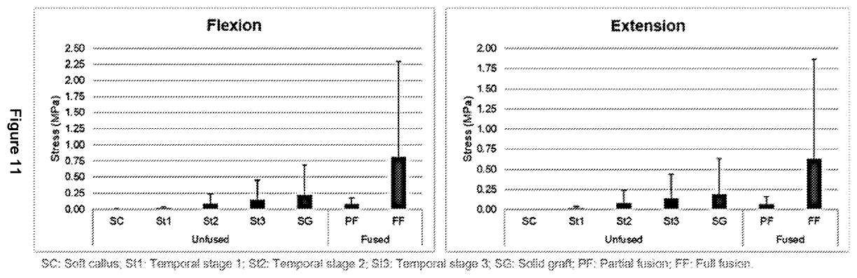

The annulus fibrosus was modelled per previously published protocols as a composite structure of concentric layers (n=4) of criss-cross collagen fibres embedded within a ground substance⁵ (FIG. 7). The ends of the fibres were rigidly anchored in the superior and inferior endplates and concentric fibre layers were connected via interlamellar bridges. Annulus fibres were modelled with varying orientation, gradually increasing from ±24° ventrally to ±46° dorsally according to published anatomical data². Ligaments were modelled as cylindrical beam elements (Table 1), with attachment and insertion sites in accordance with previous protocols and published literature^{5,6}.

TABLE 1

| | | SC | St1 | St2 | St3 | SG | PF | FF |
|---|---|---|---|---|---|---|---|---|
| Nodes | | 192,011 | 192,011 | 192,011 | 192,011 | 192,011 | 187,329 | 187,329 |
| Brick | | 1,063,748 | 1,063,748 | 1,063,748 | 1,063,748 | 1,063,748 | 1,063,748 | 1,063,748 |
| Elements | | | | | | | | |
| (4-noded | | | | | | | | |
| Tetrahedrons) | | | | | | | | |
| Facet | | 5 per joint | 5 per joint | 5 per joint | 5 per joint | 5 per joint | 5 per joint | 5 per joint |
| Articulation | | | | | | | | |
| (Nonlinear | | | | | | | | |
| Point Contact | | | | | | | | |
| Elements) | | | | | | | | |
| Annulus | | | | | | | | |
| Fibres | | | | | | | | |
| (Cylindrical | | | | | | | | |
| Beam | | | | | | | | |
| Elements) | | | | | | | | |
| | L1- | (O) 384-380- | (O) 384-380- | (O) 384-380- | (O) 384-380- | (O) 384-380- | (O) 384-380- | (O) 384-380- |
| | L2 | 362-234 (I) | 362-234 (I) | 362-234 (I) | 362-234 (I) | 362-234 (I) | 362-234 (I) | 362-234 (I) |
| | L2- | (O) 298-297- | (O) 298-297- | (O) 298-297- | (O) 298-297- | (O) 298-297- | (O) 298-297- | (O) 298-297- |
| | L3 | 298-197 (I) | 298-197 (I) | 298-197 (I) | 298-197 (I) | 298-197 (I) | 298-197 (I) | 298-197 (I) |
| | L3- | (O) 321-319- | (O) 321-319- | (O) 321-319- | (O) 321-319- | (O) 321-319- | (O) 321-319- | (O) 321-319- |
| | L4 | 318-211 (I) | 318-211 (I) | 318-211 (I) | 318-211 (I) | 318-211 (I) | 318-211 (I) | 318-211 (I) |
| | L4- | (O) 130-124- | (O) 130-124- | (O) 130-124- | (O) 130-124- | (O) 130-124- | (O) 130-124- | (O) 130-124- |
| | L5 | 102-54 (I) | 102-54 (I) | 102-54 (I) | 102-54 (I) | 102-54 (I) | 102-54 (I) | 102-54 (I) |
| | L5- | (O) 519-504- | (O) 519-504- | (O) 519-504- | (O) 519-504- | (O) 519-504- | (O) 519-504- | (O) 519-504- |
| | S1 | 466-293 (I) | 466-293 (I) | 466-293 (I) | 466-293 (I) | 466-293 (I) | 466-293 (I) | 466-293 (I) |
| Ligaments | | | | | | | | |
| per Levet | | | | | | | | |
| (Cylindrical | | | | | | | | |
| Beam | | | | | | | | |
| Elements) | | | | | | | | |
| | ALL | 14 | 14 | 14 | 14 | 14 | 14 | 14 |
| | PL | 6 | 6 | 6 | 6 | 6 | 6 | 6 |
| | TL | 16 | 16 | 16 | 16 | 16 | 16 | 16 |
| | LF | 18 | 18 | 18 | 18 | 18 | 18 | 18 |
| | ISL | 9 | 9 | 9 | 9 | 9 | 9 | 9 |
| | SSL | 4 | 4 | 4 | 4 | 4 | 4 | 4 |
| | CL | 24 per joint | 24 per joint | 24 per joint | 24 per joint | 24 per joint | 24 per joint | 24 per joint |
| | ILL | 20 | 20 | 20 | 20 | 20 | 20 | 20 |
| | LSL | 22 | 22 | 22 | 22 | 22 | 22 | 22 |

### Modelling Facet Joint Articulation

Compressive load transfer characteristics between facet joints at the bony articulating pillars were modelled by Point Contact-Tension elements (n=5 per joint) in Strand7. The contact elements were evenly distributed over the articulating faces and normally oriented.

### Loads and Boundary Constraints

A node on the anterior surface of the sacrum, below the sacral promontory, was constrained in all translational and rotational degrees of freedom. Bending moments were applied to the model using a crossbeam construct at the L1 superior endplate, mounted on a surface cap. The surface cap and crossbeam were assigned material properties of structural steel (E=200 GPa, v=0.25). A force couple was applied to the anterior and posterior extremities of the crossbeam, loading the models in flexion and extension bending. The models were loaded in a stepwise manner with pure unconstrained moments from 1 Nm to 10 Nm and solved for geometric, material, and boundary nonlinearities using the Nonlinear Static Solver in Strand7.

### Assigning Material Properties

Material properties assigned to brick, beam, and nonlinear contact elements were obtained from a previously published study with identical modelling protocols in which the values were calibrated against in vitro biomechanical testing data⁵.

### Results

### Loading of Interbody Cage

In both flexion (Fx) and extension (Ex), compressive stress on the interbody cage reduced by 20% with increasing graft stiffness from the SC to SG stage in the unfused case (Fx: 0.86 MPa (SC) to 0.69 MPa (SG); Ex: 1.01 MPa (SC) to 0.81 MPa (SG)). Cage stress increased, however, after complete bonding with both cancellous and cortical grafts (Fx: 1.47 MPa (PF), 1.22 MPa (FF); Ex: 1.53 MPa (PF), 1.31 MPa (FF)), as depicted in FIG. 8.

Stress accounts for change both in area and force. As such, change in compressive force is reported normalised to the SC bone graft model, accounting both for change in the compressive stress and change in the area under compressive stress. Progressive off-loading of the cage was observed with stiffening graft, simulating advancing fusion, from SC to SG in flexion only (St1: −18%, St2: −31%, St3: −39%, SG: −42%). Cephalad endplate bonding increased normalised force in both fused contact models (Fx: 55% (PF), 16% (FF); Ex: 47% (PF), 28% (FF)) (FIG. 9).

### Loading of Graft

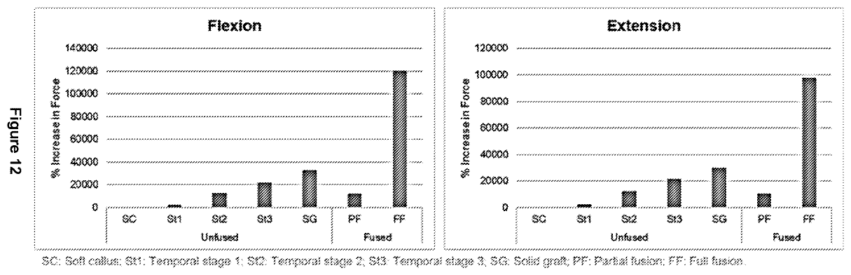

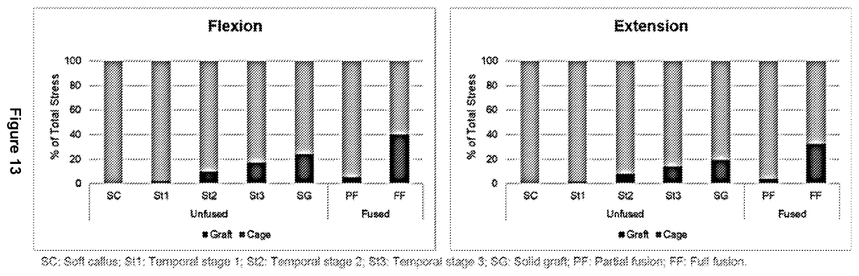

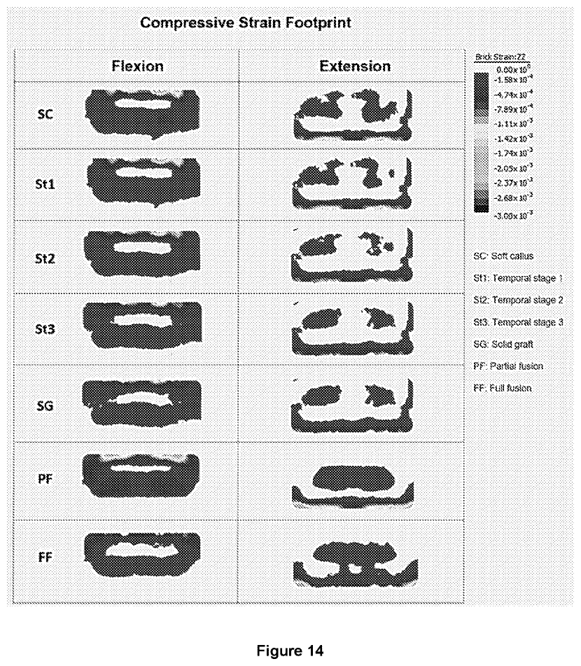

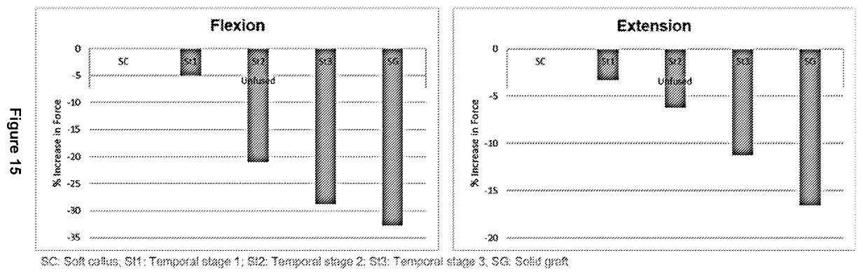

Compressive graft stress showed an increase associated with graft stiffness in flexion (SC: 0.00 MPa, St1: 0.02 MPa, St2: 0.09 MPa, St3: 0.15 MPa, SG: 0.22 MPa) and extension (SC: 0.00 MPa, St1: 0.02 MPa, St2: 0.08 MPa, St3: 0.14 MPa, SG: 0.20 MPa), shown in FIG. 11. Stress on the cancellous bone graft in the fused state was comparable to the St2 unbonded model given its similar stiffness properties (Fx: 0.08 MPa, Ex: 0.07 MPa). A similar trend was observed in normalised compressive force results (FIG. 12).

### Cage: Graft Load-Share

Increasing graft stiffness improved the compressive load-sharing between the cage and graft as a percentage of total compressive stress on the construct (FIG. 9). The SC model exhibited 99.9% stress on the cage (0.1% on graft) in forward and backward bending. The SG model showed off-loading of the cage and more stress on the graft in flexion (75.6% cage, 24.4% graft) and extension (80.4% cage, 19.6% graft). Stress-sharing between the cage and graft was associated with graft stiffness and not bonding to the endplates (Fx: 94.7% cage, 5.3% graft (PF), 60.0% cage, 40.0% graft (FF); Ex: 95.8% cage, 4.2% graft (PF), 67.3% cage, 32.7% graft (FF)).

### Cage Anterior Force

Across the unfused models, stiffening of the bone graft reduced anteriorly directed force on the cage. Anterior force decreased by 5%, 21%, 29%, and 33% respectively for St1, St2, St3, and SG in flexion compared to SC (FIG. 11). Smaller changes were noted in extension (−3% (St1), −6% (St2), −11% (St3), −17% (SG)). As with normalised compressive force, normalised anterior force accounted for both change in stress and change in area under anterior stress. The difference in contact modelling between the fused contact and unfused contact groups prevents the comparison of anterior forces between the two groups.

### Facet Axial Force

Axial force results from the facets (Table 2) represent the compressive load-transfer capabilities of the joint. In flexion, no compressive load transfer was noted in fused contact group through the L4-L5 facets. L3-L4 axial force was reduced by 11% and no significant change was observed at L5-S1. Compressive load through L4-L5 during extension was reduced by 87% due to the fused contact with no significant changes at adjacent facets.

TABLE 2

| | | Unfused | | | |
|---|---|---|---|---|---|
| | | SC ± (SD) | St1 ± (SD) | St2 ± (SD) | St3 ± (SD) |
| Level | Fx | 2.7159 ± (0.0928) | 2.7156 ± (0.0926) | 2.7143 ± (0.0922) | 2.7133 ± (0.0919) |
| Above | Ex | 12.1387 ± (3.0021) | 12.1386 ± (3.0021) | 12.1382 ± (3.0021) | 12.1379 ± (3.0020) |
| Index | Fx | 1.3372 ± (0.6038) | 1.3368 ± (0.6065) | 1.3427 ± (0.6167) | 1.3528 ± (0.6223) |
| Level | Ex | 11.0250 ± (6.5155) | 11.0223 ± (6.5136) | 11.0199 ± (6.5102) | 11.0165 ± (6.5077) |
| Level | Fx | 1.8662 ± (1.9806) | 1.8657 ± (1.9807) | 1.8644 ± (1.9813) | 1.8637 ± (1.9816) |
| Below | Ex | 4.6760 ± (2.5581) | 4.6765 ± (2.5582) | 4.6793 ± (2.5590) | 4.6813 ± (2.5596) |

| | | Unfused | Fused | |
|---|---|---|---|---|
| | | SG ± (SD) | PF ± (SD) | FF ± (SD) |
| Level | Fx | 2.7125 ± (0.0916) | 2.4261 ± (0.0000) | 2.4200 ± (0.0024) |
| Above | Ex | 12.1376 ± (3.0020) | 11.8734 ± (2.8486) | 11.8713 ± (2.8478) |
| Index | Fx | 1.3650 ± (0.6266) | 0.0000 ± (0.0000) | 0.0000 ± (0.0000) |
| Level | Ex | 11.0125 ± (6.5055) | 1.4474 ± (1.0534) | 1.3695 ± (0.9890) |
| Level | Fx | 1.8632 ± (1.9818) | 2.0048 ± (1.9506) | 1.9982 ± (1.9532) |
| Below | Ex | 4.6829 ± (2.5600) | 4.7204 ± (2.5774) | 4.7267 ± (2.5791) |

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

1. White AA, Panjabi MM. Clinical biomechanics of the spine. 2nd ed. Philadelphia, PA: JB Lippincott; 1990.
2. Schmidt H, Heuer F, Simon U, et al. Application of a new calibration method for a three-dimensional finite element model of a human lumbar annulus fibrosus. Clin Biomech (Bristol, Avon). 2006;21(4):337-344.
3. Denoziere G, Ku DN. Biomechanical comparison between fusion of two vertebrae and implantation of an artificial intervertebral disc. J Biomech. 2006;39(4):766-775.
4. Roberts S, Menage J, Urban JP. Biochemical and structural properties of the cartilage end-plate and its relation to the intervertebral disc. Spine (Phila Pa 1976). 1989;14(2):166-174.
5. Ramakrishna VAS, Chamoli U, Viglione LL, Tsafnat N, Diwan AD. The Role of Sacral Slope in the Progression of a Bilateral Spondylolytic Defect at L5 to Spondylolisthesis: A Biomechanical Investigation Using Finite Element Analysis. Global Spine J. 2018;8(5):460-470.
6. Behrsin JF, Briggs CA. Ligaments of the lumbar spine: a review. Surg Radiol Anat. 1988;10(3):211-219.

1. A surgical implant comprising:
one or more sensor elements disposed in and/or on the surgical implant and adapted to measure a force exerted on the surgical implant when implanted in a subject;
a transmitter operably coupled to the one or more sensor elements and adapted to receive a mechanical data representative of the force therefrom and transmit said mechanical data to an external receiver;
a power supply operably coupled to the one or more sensor elements and the transmitter, the power supply adapted to harness energy wirelessly.

2. The surgical implant of claim 1, wherein the one or more sensor elements are selected from the group consisting of a piezoelectric sensor, a piezoresistive sensor, a capacitive sensor and any combination thereof.

3. The surgical implant of claim 1, wherein the one or more sensor elements are operably coupled to a printed circuit board (PCB).

4. The surgical implant of claim 3, wherein the PCB is a thin or flexible PCB or a rigid PCB.

5. The surgical implant of claim 1, wherein the one or more sensor elements comprise one or more capacitive sensors not operably coupled to a PCB.

6. The surgical implant of claim 5, wherein the one or more capacitive sensors comprises a biocompatible substrate, such as Polyether-ether-ketone (PEEK), Bioactive glass, polydimethylsiloxane (PDMS), polyimide (PI) or combinations including range of implantable composites.

7. The surgical implant of claim 1, wherein the one or more sensor elements, the transmitter and/or the power supply are disposed entirely within the surgical implant.

8. The surgical implant of claim 1, wherein the one or more sensor elements, the transmitter and/or the power supply are disposed at least partly on one or more outer surfaces of the implant.

9. The surgical implant of claim 1, further comprising a processing unit operably coupled to the transmitter and the one or more sensor elements, wherein the processing unit is adapted to process the force measured by the one or more sensor elements into the mechanical data transmittable by the transmitter.

10. The surgical implant of claim 9, wherein the processing unit is disposed in and/or on the surgical implant.

11. The surgical implant of claim 1, wherein the power supply is adapted to receive energy wirelessly from an external power source or is adapted to harvest energy from a subject in which the surgical implant is implanted.

12. The surgical implant of claim 11, wherein the power supply is at least partly inductively powered.

13. The surgical implant of claim 12, wherein the surgical implant is able to be activated from an inactive state to an active state by positioning of the external power source proximate or adjacent thereto.

14. (canceled)

15. The surgical device of claim 1, wherein the power supply does not include an incorporated power source, such as a battery unit.

16. The surgical device of claim 1, wherein the surgical implant is or comprises a fracture fixation device or a joint implant.

17. The surgical device of any one of the preceding claims claim 1, wherein the surgical implant is a spinal implant, such as an interbody fusion cage.

18. (canceled)

19. A surgical implant system, the system comprising:
(a) a surgical implant comprising:
one or more sensor elements disposed in or on the surgical implant and adapted to measure a pressure and/or a strain exerted on the surgical implant when implanted in a subject;
a transmitter operably coupled to the one or more sensor elements and adapted to receive pressure and/or a strain data therefrom and transmit said data to an external receiver; and
a power supply operably coupled to the one or more sensor elements and the transmitter, the power supply adapted to receive or harvest energy wirelessly; and
(b) a control unit comprising a receiver adapted to receive the pressure and/or a strain data from the transmitter of the surgical implant.

20.-22. (canceled)

23. A method of monitoring a surgical implant in a subject, said method including the steps of:
(a) obtaining a mechanical data from one or more sensor elements that are disposed in or on the surgical implant and adapted to measure a force exerted thereon;
(b) transmitting the mechanical data by a transmitter operably coupled to the one or more sensor elements and adapted to receive data therefrom to a receiver external to the surgical implant and the subject; and
(c) powering the one or more sensor elements and the transmitter with a power supply operably coupled thereto, wherein the power supply is adapted to receive or harvest energy wirelessly.

24.-37. (canceled)

38. A non-transitory computer-readable storage medium whose stored contents configure a computing system to perform the method of claim 23.

39. The method of claim 23, further including the step of processing the mechanical data to determine: (i) a degree of healing of a bone fracture in the subject, wherein the bone fracture is fixed with the surgical implant; (ii) a degree of fusion of two adjacent vertebrae in the subject, wherein the surgical implant is positioned between the two adjacent vertebrae; (iii) the integrity, alignment and/or positioning of the surgical implant in the subject; or (iv) the force exerted on the surgical implant during movement of a joint previously implanted with the surgical implant.
